(19) United States
(12) Patent Application Publication
(10) Pub. No.: US 2006/0134222 A1
(43) Pub. Date: Jun. 22, 2006

(54) Process for encapsulating an active lipid-soluble substance by preparing a pit emulsion and emulsion obtained

(76) Inventor: Luc Jugla, Villemoirieu (FR)

Correspondence Address: OLIFF & BERRIDGE, PLC, P.O. BOX 19928, ALEXANDRIA, VA, 22320, US

(21) Appl. No.: 10/537,811

(22) PCT Filed: Dec. 24, 2003
(86) PCT No.: PCT/FR03/03900

§ 371 Date: Oct. 28, 2005

(30) Foreign Application Priority Data
Dec. 26, 2002 (FR) 02/16723

(51) Int. Cl. A61K 9/50; B01J 13/04

(52) U.S. Cl. 424/490; 977/906; 264/4.1

(57) ABSTRACT

The invention relates to a method of encapsulating an active lipid-soluble substance in nanocapsules, by preparing an emulsion. The inventive method is characterised in that it consists in: (a) obtaining an aqueous phase and an oil phase; (b) raising the temperature of the two phases to a temperature greater that the phase inversion temperature; (c) mixing the two phases; (d) incorporating the active lipid-soluble substance into the lipid-soluble phase; (e) allowing the temperature to decrease to the phase inversion temperatures; (f) once the phase inversion is effective and the emulsion is in a continuous aqueous phase, quenching the emulsion obtained in order to lower the temperature thereof. The invention also relates to the emulsion that can be obtained using the inventive method, said emulsion being characterised in that the average nanocapsule size is less than 300 nm.

The present invention relates to the field of vectorizing active principles.

The efficacy of a formulation both in pharmacy and in cosmetics depends on the active principles but also on their release system, and many vectorization means have been explored either in cosmetics or in pharmacy.

Among these, mention may be made of nanoparticles. Nanoparticles are colloidal particles ranging from 1 to 1000 nm in size. They are macromolecules in which the active principle is dissolved, trapped or encapsulated. These nanoparticles refer to very different systems, for instance nanospheres and nanocapsules, which are, respectively, matrix systems for the nanospheres, and reservoir systems for the nanocapsules.

Nanospheres are solid matrix particles in which the active principle is finely dispersed in the polymer matrix.

Nanocapsules are particles consisting of a core that is liquid or semiliquid at room temperature, which contains the active principle, coated with a film that is solid at room temperature.

The present invention more particularly relates to the field of vectorizing liposoluble active principles in a reservoir system of nanocapsule type. Nanocapsules are aqueous suspensions of small vesicles (generally between 100 and 400 nm), the thin rigid wall of which consists of macromolecules of natural, synthetic or semisynthetic origin. These systems allow the encapsulation in the lipophilic core of relatively large amounts of active principles, which are usually lipophilic, and may be obtained either via polymerization reactions or from preformed polymers. Many processes for formulating nanocapsules by emulsification are described, and examples that will be mentioned include the processes described in patents U.S. Pat. No. 5,079,322 or EP 0 717 989, for obtaining emulsions incorporating liposoluble active principles. The term “liposoluble active principles” in particular means any chemical compound or mixture that is soluble in oily substances used in cosmetics, the food sector, pharmaceuticals or the veterinary sector or any compound that is advantageous as a result of its properties. Some liposoluble active principles are sensitive to exposure to temperatures above 50° C., and sensitive to light and to oxidation. One of the solutions currently used for vectorizing these active principles is to formulate them in emulsions. However, on account of their instability, when these liposoluble active principles are used in emulsified systems, they are introduced at the end of the process into an oil-in-water emulsified system at a temperature below 50° C., for example, and they then become randomly distributed, particularly in the aqueous phase and will then be partially destroyed by the surrounding medium.

These processes are therefore not entirely satisfactory, either because the amounts of active principles incorporated are insufficient to achieve the desired activities, or because the stability is not correct, or even because the production processes are difficult to implement industrially.

To improve these formulations, processes of emulsification by phase inversion, known as “PIT (Phase Inversion Temperature) emulsion”, for instance those described in patents WO 20011975, EP 1 093 795 or WO 200071676 for obtaining oil-in-water emulsions containing an active principle, have been proposed.

These processes include the incorporation, for example, of an active principle into an oily phase, the addition of some of the aqueous phase to the mixture obtained, heating with stirring to a temperature above the phase inversion temperature, addition of the remainder of the aqueous phase, and cooling. For example, WO 200164328 discloses a process for preparing lipid nanocapsules based on the phase inversion of an oil/water emulsion induced by several cycles of raising and reducing the temperature. The emulsions obtained are very fine and do not require homogenization steps. These processes allow the production of very fine dispersions of the emulsion (0.1 to 0.3 μm) and great stability, since, during the phase inversion, the interface tension is minimal and allows very fine droplets to be obtained. However, the phases of temperature increase to obtain the phase inversion, which may optionally be repeated, are incompatible with the formulation of active principles liable to undergo physical or chemical degradation due to excessive exposure to a temperature above 50° C.

In the present invention, the lipid nanocapsules are formulated via a process of emulsification by phase inversion induced by passing the emulsion above the phase inversion temperature, but which allows the active principle to be preserved by incorporating it into the oily continuous phase, and thus without contact with the aqueous phase, above the phase inversion temperature.

Specifically, the incorporation of the liposoluble active principle into the formulation, at a temperature above the phase inversion temperature, i.e. when the emulsion is in the oily continuous phase (water-in-oil emulsion), makes it possible to obtain a good distribution of the active principle in the oily phase, limits its contact with the aqueous phase, and, surprisingly, although the temperature is high, since the residence time at this temperature is very short because this incorporation is followed by annealing of the emulsion, the degradation phenomena are limited or eliminated.

The present invention relates to a process for encapsulating a liposoluble active principle in nanocapsules by preparing an emulsion, characterized in that:
a) an aqueous phase and a fatty phase are provided,
b) the temperature of the two phases is raised to a temperature above the phase inversion temperature,
c) the two phases are mixed together,
d) the liposoluble active principle is incorporated into the liposoluble phase,
e) the temperature is lowered to the phase inversion temperature,
f) once the phase inversion is effective and the emulsion is in the aqueous continuous phase, the emulsion obtained is annealed to lower its temperature.

In one variant after step c), a step c′) is performed, which consists in lowering the temperature to a temperature immediately above the phase inversion temperature before incorporating the active principle.

This lowering of temperature may be induced or may take place naturally.

In one variant, the temperature may be left to lower naturally or the temperature may be lowered to a desired temperature by performing an annealing operation.

The invention also relates to a process according to the claim, characterized in that step c) is performed before step b). In this process variant, the mixing of the two phases is performed before raising the temperature or during the raising of temperature, but before the temperature reaches the phase inversion temperature. The emulsion obtained is then brought to a temperature above the phase inversion temperature, and the active principle is then incorporated.

In one variant of the process according to the invention, the emulsion obtained is then concentrated by withdrawal of some of the aqueous phase.

Advantageously, this concentration step may be performed by tangential ultrafiltration.

According to the invention, the “annealing” step f) is performed by adding an additional amount of aqueous phase brought to a temperature at least below the phase inversion temperature, and optionally below room temperature. This sudden and rapid cooling step makes it possible to lower the temperature of the emulsion and to reduce the time of exposure of the active principle to a raised higher temperature.

This annealing may also be performed using a heat-exchange cooling system or by adding liquefied gas, for example nitrogen.

The term “temperature immediately higher than the phase inversion temperature” means a temperature a few degrees higher, in practice 1 or 2° C. higher than the phase inversion temperature, the phase inversion temperature of the system having been determined experimentally beforehand by monitoring the conductivity of the system or by visual observation.

Among the active principles that may be encapsulated via this process, mention will be made more particularly of “unstable” liposoluble active principles, i.e. active principles liable to degrade if they are exposed to temperatures above 40° C. for longer than 30 minutes, or active principles that are sensitive to oxidation due to the presence of water in the formulation, or that are degraded by pH variations, UV radiation or the presence of products liable to cause side reactions with said active principles.

Among the liposoluble active principles that may be encapsulated via this process, examples that will be mentioned include:
liposoluble vitamins and derivatives thereof, such as the retinoid family (retinol, retinaldehyde and retinoic acid), the carotenoid family, and tocopherol and its derivatives,
polyphenols such as flavonoids (e.g.: isoflavonoids, quercetin), stilbenes (e.g.: resveratrol), catechins (e.g.: epicatechin 3-galate, epigallocatechin 3-gallate),
fragrance components, for instance vanillin, indole, and more generally essential oils such as essential oils of citrus fruit or of lavender,
liposoluble pharmaceutical active principles such as: fluvastatin, ketoprofen, verapamil, atenolol, griseofulvin, ranitidine.

In the process according to the invention, the emulsion comprises from 5% to 30% of fatty substance constituting the fatty phase and from 45% to 92% of water constituting the aqueous phase. The proportion of the fatty phase relative to the aqueous phase associated therewith depends on the amount of active principle to be encapsulated and on the type of emulsion. The proportion of fatty phase may also have an influence on the size of the nanocapsules obtained.

The constituents of the fatty phase may be chosen from paraffin derivatives or more or less complex triglycerides. The choice of these constituents will depend on the nature of the lipophilic active principle to be encapsulated, but also on their potential influence on the phase inversion temperature, or even on their influence on the size of the nanocapsules obtained.

The nature of the active principle to be encapsulated will have an influence on the choice of constituents of the fatty phase, since the constituents will be selected as a function of:
the potential solubility of the active principle in this phase,
their neutrality with respect to the active principle, i.e. they must not be oxidizing with respect to the active principle, i.e. they must have a low acid number, must not be acidic and must have a low iodine number,
their compatibility with a phase inversion emulsification technique,
their ability to give the lowest possible phase inversion temperature.

When the phase inversion temperature is too high, ingredients capable of lowering this phase inversion temperature will be added to the medium.

Specifically, the more pronounced lipophilic nature of certain constituents liable to be chosen on account, for example, of their ability to dissolve the active principles may lead to an increase in the phase inversion temperature, since the enhancement of the hydrophobic bonds between the surfactant and the oil leads to an increase in the energy required to invert the system. The polarity of the constituents of the fatty phase also has an influence on the phase inversion temperature: the more polar the constituents, the higher the phase inversion temperature. On the other hand, saturated constituents, with the lowest possible iodine number, are capable of reducing the phase inversion temperature.

Although the residence time at a temperature above the phase inversion temperature is extremely short, it will nevertheless be sought to formulate emulsions whose phase inversion temperature is as low as possible.

The constituents of the fatty phase will thus preferably be chosen from mineral oils or mineral oil substitutes such as isohexadecane, silicones, especially cyclomethicones or polydimethylsiloxane, C8 to C12 triglycerides, for example capric and caprylic acid triglycerides, and mixtures thereof.

The choice of the emulsifying system is also an important criterion that has an influence on the stability of the emulsions obtained and on the particle size. Two values characterize an emulsifying system, the lipophilic surfactant/hydrophilic surfactant ratio (LS/HS ratio) and the overall percentage of surfactants.

The emulsifying systems used in the present invention will be chosen from systems whose LS/HS ratio is between 1/1 and 1/50. The percentage of water-soluble surfactant will preferably be between 2% and 10% and the percentage of lipophilic surfactant will preferably be between 1% and 5%.

The water-soluble surfactants are especially chosen from glycol esters, glycerol esters, itol esters, sorbitan esters and polyethylene glycol esters. Among the polyethylene glycol esters that will especially be chosen are those whose carbon-based chain is between 10 and 22 carbon atoms and for which the number of polyethylene glycol monomers is between 5 and 30. These water-soluble surfactants may also be chosen from fatty alkyl ethers of polyethylene glycol, whose fatty alcohol is chosen from those containing from 10 to 22 carbon atoms and whose monomer number is between 5 and 30.

Lipophilic surfactants will also be added to the mixture; these surfactants are characterized by their ability to give W/O emulsions when used as emulsifiers alone or predominantly. Among these emulsifiers, mention will be made of monoglycerol esters and polyglycerol esters of fatty acids, silicone emulsifiers such as cetyl dimethicone copolyol, and polyhydroxystearic acid esters of polyethylene glycol.

According to one embodiment of the invention, the salt may be added to the aqueous phase. It has been demonstrated that the addition of salt reduces the interaction between the polar groups and the water and reduces the hydrophilicity of the surfactant, and thus the CMC. In addition, it produces a screen effect that facilitates approach between the polar groups.

Moreover, studies have revealed that modification of the salt concentration results in a displacement of the phase inversion zone. The higher the salt concentration, the lower the phase inversion temperature.

Other constituents may be added to one or other of the phases; examples that will be mentioned include preserving agents for preventing the growth of certain microorganisms in the aqueous phase.

The antioxidants are added to the system to prevent impairment of certain readily oxidizable compounds in the lipid phase. They are chosen, for example, from the group consisting of butylhydroxyanisole (BHA), butylhydroxytoluene (BHT), propyl gallate, α-tocopherol and EDTA. These antioxidants will be used in concentrations ranging from 0.01% to 3%; for example, BHT will be used in concentrations ranging from 0.01% to 1%, α-tocopherol in concentrations ranging from 0.1% to 3% and EDTA in concentrations ranging from 0.05% to 2%.

In the process according to the invention, the stirring speed will be between 100 and 3000 rpm. Specifically, during the emulsification, a dynamic equilibrium is established between rupture (zones at high shear) and coalescence (zones at low shear). The stirring speed affects the rupture and the coalescence, and this stirring speed will thus have an influence on the size distribution and the stability of the emulsion.

In the process according to the invention, detection of the phase inversion is performed:
either by visualization of the formulation: the organization of the system in the form of nanoparticles is reflected visually by a change in the appearance of the initial system, which goes from opaque-white to translucent-white. For poorly dispersed emulsions, the appearance occasionally becomes bluish during the phase inversion,
or by measuring the conductivity, which increases when the emulsion passes from a water-in-oil system to an oil-in-water system.

Specifically, the conductivity increases when the emulsion passes from a water-in-oil system to an oil-in-water system. An electrolyte-rich aqueous continuous phase is characterized by a high conductivity value. The PIT zone is defined as being a zone in which the conductivity of the medium changes from a zero value (characterizing an oily continuous phase) to a value of a few μs/cm. This change takes place over a temperature range known as the PIT zone.

The particle diameter is measured via an optical method of light measurement known as light scattering, which is based on various physical and mathematical laws including PCS (Photon Correlation Spectroscopy). The principle of the measurement may be described as a study of the speed of particles subjected to Brownien motion, the small particles vibrating considerably and moving quickly, whereas those of larger diameter vibrate little and move more slowly. The interaction of a light beam with the particles makes it possible, after mathematical modeling, to estimate the particle diameter.

The present invention also relates to lipid nanocapsules obtained via the process according to the invention, the mean size of which is less than 300 nm and preferably on average 150 nm.

Emulsions according to the invention are described below.

### EXAMPLE 1

A fatty phase containing the following ingredients is formulated:

| | |
|---|---|
| tocopheryl acetate (vitamin E acetate) | 0.5% |
| glyceryl stearate and ceteareth-12 and ceteareth-20 and cetearyl | 3% |
| alcohol (Emulgade SEV) | |
| ceteareth-20 (Eumulgin B2) | 2% |
| isohexadecane (Arlamol HD) | 6% |
| cyclomethicone (Dow Corning 345) | 3% |
| butylhydroxytoluene (BHT) | 0.1% |

An aqueous phase containing the following ingredients is formulated:

| | |
|---|---|
| sodium salt of EDTA (BASF (disodium EDTA)) | 0.5% |
| demineralized water | 25% |

The two phases formulated above are heated to a temperature of 85° C.

The two phases are combined by adding the aqueous phase to the fatty phase with shearing stirring at 700 rpm.

The active principle retinol, as a 7% solution in a caprylic acid triglyceride, is then incorporated into the emulsion obtained by mixing together the aqueous phase and the fatty phase at a temperature in the region of 81° C.

The phase inversion takes place at 73° C., this phase inversion being detected by an increase in the conductivity of greater than 1 μS/cm.

An additional aqueous phase containing a preserving agent, Glydant Plus Liquid (DMDM hydantoin and iodopropynyl butylcarbamate (sold by the company Lonza Inc. (0.5%) and water 51.9% is rapidly incorporated into the emulsion obtained above containing the retinol.

The emulsion may then be concentrated by tangential ultrafiltration.

### EXAMPLE 2

According to the same procedure as in example 1, an emulsion is prepared starting with the following phases:

Fatty Phase:

| | |
|---|---|
| PEG-30 dipolyhydroxystearate | 2% |
| PEG-6 stearate and ceteth-20 and steareth-20 | 6% |
| isohexadecane | 6% |
| cyclomethicone | 3% |
| tocopheryl acetate | 0.5% |
| butylhydroxytoluene | 0.1% |

Aqueous Phase:

| | |
|---|---|
| disodium EDTA | 0.2% |
| demineralized water | 25% |

Active Principle:
retinol, as a 7% solution in a caprylic acid triglyceride

The phase inversion takes place at 71° C.

Additional Aqueous Phase:

| | |
|---|---|
| chlorhexidine digluconate | 0.5% |
| water | 49.7% |

### EXAMPLE 3

According to the same procedure as in example 1, an emulsion is prepared from the following phases:

Fatty Phase:

| | |
|---|---|
| PEG-30 dipolyhydroxystearate | 2% |
| PEG-6 stearate and ceteth-20 and steareth-20 | 6% |
| isohexadecane | 6% |
| cyclomethicone | 3% |
| tocopheryl acetate | 0.5% |
| butylhydroxytoluene | 0.1% |
| caprylic/capric triglyceride | 6% |

Aqueous Phase:

| | |
|---|---|
| disodium EDTA | 0.2% |
| demineralized water | 25% |

Active Principle:
retinol, as a 0.33% solution in a caprylic acid triglyceride

The phase inversion takes place at 80° C.

Additional Aqueous Phase:

| | |
|---|---|
| chlorhexidine digluconate | 0.5% |
| sodium methylparaben | 0.2% |
| water | 50.17% |

Among the advantages of the process according to the invention, mention may be made of the size of the droplets obtained, of less than 300 nm, which has the following advantages:
improved bioavailability of the incorporated active principle, since the penetration of the emulsion is promoted by the minimal size of the particles encapsulating the active principle, this improved bioavailability of the incorporated active principle allows the final concentration in the product to be lower than with standard encapsulating systems and reduces the possible side effects,
better physical stability of the finished product; specifically, the smaller the particle size, the more physically stable the system on account of the disappearance of the maturation and coalescence phenomena,
the production of monodisperse systems (polydispersity index<0.25): since the size of nanocapsules is homogeneous, the Oswald maturation is limited,
manufacturing processes that are faster and more economical than the standard emulsification processes on account of the reduction in the energy required.

1-8. (canceled)

9. A process for encapsulating a liposoluble active principle in nanocapsules by preparing an emulsion, characterized in that:
a) an aqueous phase and a fatty phase are provided,
b) the temperature of the two phases is raised to a temperature above the phase inversion temperature,
c) the two phases are mixed together,
d) the liposoluble active principle is incorporated into the liposoluble phase,
e) the temperature is lowered to the phase inversion temperature,
f) once the phase inversion is effective and the emulsion is in the aqueous continuous phase, the emulsion obtained is annealed to lower its temperature.

10. The process as claimed in claim 9, characterized in that step c′) is performed, which consists in lowering the temperature to a temperature immediately above the phase inversion temperature before incorporating the active principle.

11. The process as claimed in claim 9, characterized in that step c) is performed before step b).

12. The process as claimed in claim 9, characterized in that the emulsion obtained is then concentrated by withdrawal of some of the aqueous phase.

13. The process as claimed in claim 9, characterized in that step e) is performed by adding an additional amount of aqueous phase brought to a temperature below the phase inversion temperature.

14. The process as claimed in claim 9, characterized in that the active principle is dissolved in an additional amount of fatty phase before being incorporated into the system.

15. The process as claimed in claim 9, characterized in that the active principle is chosen from the group consisting of liposoluble vitamins such as retinol, retinoids, vitamin E and carotenoids, polyphenols and fragrance components.

16. An emulsion that may be obtained via a process as claimed in claim 9, characterized in that the size of the nanocapsules is on average less than 300 nm.
